(19) United States
(12) Patent Application Publication
(10) Pub. No.: US 2026/0294248 A1
(43) Pub. Date: Oct. 1, 2026

(54) SYSTEM AND METHOD FOR DETERMINING A CARDIOMYOCYTE MANGANESE ION UPTAKE RATE CONSTANT TO INDICATE A T-TUBULE DENSITY

(71) Applicant: IC TARGETS AS, Oslo (NO)

(72) Inventor: Jonny ØSTENSEN, Oslo (NO)

(21) Appl. No.: 19/489,936

(22) PCT Filed: Jun. 12, 2024
(86) PCT No.: PCT/NO2024/050137

§ 371 Date: Dec. 4, 2025

(30) Foreign Application Priority Data
Jun. 16, 2023 (NO) 20230696

(51) Int. Cl. A61B 5/00; A61B 5/055; A61B 5/145; G01R 33/50; G01R 33/56; G06T 7/00

(52) U.S. Cl. CPC A61B 5/0044; A61B 5/055; G01R 33/50; G01R 33/5601; G06T 7/0012; A61B 5/14535; A61B 2576/023; G06T 2207/10088; G06T 2207/30048

(57) ABSTRACT

A method, CRM and system for determining a cardiomyocyte manganese ion uptake rate constant in one or more regions of interest in a heart of a subject comprising: a providing step comprising: providing a left ventricular myocardial image slice covering one or more regions of interest taken during a first time period; providing one or more longitudinal relaxation time weighted images of the left ventricular blood pool taken during the first time period to measure an average signal intensity in blood during the first time period; providing a hematocrit measurement; providing an extracellular volume measurement for each of the one or more regions of interest; an administration step comprising administering a contrast agent comprising manganese 2+ ions; an image acquisition step comprising: performing image acquisition of the left ventricular blood pool using a longitudinal relaxation time weighted pulse sequence at a plurality of time intervals during a second time period to measure respective signal intensities in blood at the plurality of time intervals; performing a longitudinal relaxation time map image acquisition of a left ventricular myocardial image slice covering the one or more regions of interest during a third time period; a measurement step comprising measuring a signal intensity in blood at each of the plurality of time intervals using an image taken at a time point within said corresponding time interval; and a calculation step comprising: calculating a change in a myocardial longitudinal relaxation rate for each region of interest from a baseline myocardial longitudinal relaxation rate extracted from the myocardial image slice taken during the first time period and a myocardial longitudinal relaxation rate at an end time point of an MRI examination extracted from the myocardial image slice during the third time period; calculating a blood longitudinal relaxation rate for each of the plurality of time intervals using respective signal intensities in blood at the plurality of time intervals and a relationship between signal intensity and said plurality of relaxation rates using the integral over all the changes in blood longitudinal relaxation rate in conjunction with the myocardial longitudinal relaxation rate at the end time point in a proportionality relationship with a cardiomyocyte Mn2+ uptake rate constant to estimate the cardiomyocyte Mn2+ uptake rate constant for each of the one or more regions of interest; wherein said first time period is before an administration of the contrast agent, said second time period is during and after an administration of the contrast agent, and said third time period encompasses the end timepoint of an MRI examination.

### FIELD OF INVENTION

The present invention relates to manganese-enhanced magnetic resonance imaging (MRI), more specifically to a system and a method for determining the cardiomyocyte manganese uptake rate constant of a subject, using a manganese-based contrast agent, and thereby providing an indication of T-tubule density.

### BACKGROUND

Cardiomyocyte manganese uptake rate refers to the rate at which cardiomyocytes, which are the muscle cells of the heart, take up manganese 2+ ions from their surrounding environment.

The uptake of manganese by cardiomyocytes is facilitated by specific transporters located on a cell membrane. The rate of manganese uptake can be influenced by several factors, including a concentration of manganese in the extracellular environment, an activity of the transporters, a density of T-tubuli in which the transporters are located, and the overall metabolic state of the cardiomyocytes.

The uptake rate of manganese by cardiomyocytes can be measured using experimental techniques such as radiotracer studies using for example ^{52g}Mn or ^{52m}Mn in PET imaging [Brandt et al. 2019, Atkins 1979] or fluorescence imaging. Radiotracer studies involve using radioactive manganese isotopes such as ^{52g}Mn, ⁵⁴Mn or ^{52m}Mn [Atkins 1979] and monitoring their entry into the cells via L-type calcium channels over a specific time period. By analysing the data obtained from these experiments, researchers can quantify the rate of manganese uptake and understand how it is affected by different factors. Fluorescent imaging to quantify the T-tubule density can only be done ex vivo on tissue samples [Frisk et al 2021].

Manganese is a calcium analogue and understanding the cardiomyocyte manganese uptake rate is important for elucidating the role of calcium in cardiac health and disease. It provides insights into the mechanisms underlying calcium homeostasis in the heart.

Cardiac MRI is a non-invasive diagnostic tool that is commonly used to evaluate the structure and function of the heart. The most common use of cardiac MRI is to assess the presence and severity of conditions such as: coronary artery disease, cardiomyopathy, heart failure, congenital heart disease, and valvular heart disease, to name a few non-limiting examples.

Contrast agents can add information to an MRI examination that cannot be obtained with unenhanced MRI. As a result of its high paramagnetic moment, manganese, in the form of Mn²⁺ ions, was one of the first MRI contrast agents to be assessed in humans. Manganese-enhanced MRI (MEMRI) directly provides intracellular enhancement of viable myocardium and is under investigation as a useful intracellular myocardial contrast enhancement method for identifying functional myocardium [WO2008087445A3, EP2117608B1]. Myocardial MEMRI relies upon two important properties of the Mn²⁺ ion.

Firstly, the Mn²⁺ ion diffuses rapidly from plasma into an extracellular space, containing extracellular fluid, and T-tubuli where it enters the cardiomyocytes, via voltage gated L-type calcium channels (LTCCs) accumulating in the cell in proportion to the LTCC calcium influx. More accurately the extracellular space can be defined as an extracellular volume.

Secondly, since the Mn²⁺ ion is paramagnetic, a longitudinal relaxation time (T1) in MRI will be shortened, hence functioning as an efficient T1 contrast agent. For quantitative measurements of the contrast effect, the longitudinal relaxation rate (R1) is used since a change in R1 is directly proportional to a change in the concentration of the contrast agent, i.e., the R1 is the inverse of T1 as shown in equation 1.

$$R 1 = \frac{1}{T 1} \qquad ( 1 )$$

With reference to FIG. 1, unchelated Mn²⁺ is mainly bound to plasma proteins. However, Mn²⁺ may be in steady state with a low concentration of free Mn²⁺ 106 which can rapidly diffuse into the extracellular space 108, containing the extracellular fluid (ECF), and the T-tubuli 110 where it enters the cardiomyocytes through the LTCCs 112 each time the channels are opened by an action potential. Unlike a calcium 2+ ion (Ca²⁺) 114, which has a significant redistribution between intracellular and extracellular compartments during the course of each heartbeat, Mn²⁺ accumulates in the cardiomyocytes, where it binds to intracellular proteins 116.

Thus, an uptake of Mn²⁺ in the cardiomyocytes is irreversible in nature.

Allowing indirect assessment of myocardial calcium influx through the LTCC, which is proportional to the T-tubule density, the Mn²⁺ ion is useful both as an imaging contrast agent and a physiological and/or metabolic tracer, and may provide information about the local T-tubule density in the heart during stress and heart disease.

The observed change in R1 in the myocardium (ΔR1_{MYO}) during an MRI scan after injection of a Mn²⁺ based contrast agent is the sum of an R1 change in the Extracellular Fluid (ΔR1_{ECF}) and in an R1 in a cellular tissue as shown in equation 2.

$$Δ R {1}_{M Y O} = Δ R {1}_{E C F} + Δ R {1}_{C E L L S} \qquad ( 2 )$$

The change in R1 in the ECF (ΔR1_{ECF}) is proportional to the extracellular volume (ECV) and the change in R1 in plasma (ΔR1_{PLASMA}) with a proportionality constant k_{ECF}.

$$Δ R {1}_{E C F} = {k}_{E C F} * E C V * Δ R {1}_{P L A S M A} \qquad ( 3 )$$

For rapidly diffusing agents, such as free Mn²⁺, k_{ECF} is very close to 1. Due to the irreversible nature of the uptake of Mn²⁺ in the cardiomyocytes, the change in R1 in the cellular tissue fraction (ΔR1_{CELLS}) is proportional to a cellular volume fraction, which is approximately (1−ECV), and an integral of the change in R1 in the ECF (ΔR1_{ECF}) with a proportionality constant k_{CELL} as shown in equation 4.

$$Δ R {1}_{C E L L S} = {k}_{C E L L} * ( 1 - E C V ) * \int \frac{Δ R {1}_{E C F}}{E C V} \qquad ( 4 )$$

k_{CELL} is a rate constant for an influx of Mn²⁺ into corresponding cells. Combining the equations 2, 3 and 4 gives a relationship between the change in R1 in myocardial tissue (ΔR1_{MYO}) and the change in R1 in plasma (ΔR1_{PLASMA}) as shown in equation 5.

$$Δ R {1}_{M Y O} = {k}_{E C F} * ( E C V * Δ R {1}_{P L A S M A} + {k}_{C E L L} * ( 1 - E C V ) * \int Δ R {1}_{P L A S M A} ) \qquad ( 5 )$$

When the ECF and plasma are in dynamic equilibrium with respect to highly diffusible Mn²⁺ ions, k_{ECF}≈1 and equation 5 can be simplified to equation 6.

$$Δ R {1}_{M Y O} = E C V * Δ R {1}_{P L A S M A} + {k}_{C E L L} * ( 1 - E C V ) * \int Δ R {1}_{P L A S M A} \qquad ( 6 )$$

Previous techniques, such as those submitted by Patlak [Clifford S. Patlak et al. Graphical Evaluation of Blood-to-Brain Transfer Constants from Multiple-Time Uptake Data; Journal of Cerebral Blood Flow and Metabolism; 3:1-7; 1983] originally proposed to use the slope of a linear regression line with

$$\frac{\int Δ R {1}_{P L A S M A}}{\int Δ {R1}_{P L A S M A}}$$

as X axis and

$$\frac{Δ R {1}_{M Y O}}{Δ R {1}_{P L A S M A}}$$

as Y axis as a measure of irreversible tissue uptake rate constant. However, this method does not take into account a partial volume of the irreversible tissue fraction, (1−ECV), and leads to a systematic underestimate of the cellular uptake rate constant. The previous technique also proposed to use the intercept of the regression line as a measure of the ECV, which often may lead to impossible values outside the range 0.0 to 1.0 without any physiological meaning. Another method for estimating the cellular uptake rate constant was proposed by Østensen et al. in [WO 2022/258517 A1] by introducing the cellular partial volume estimate (1−ECV) in the method.

However, these and all other methods described in the prior art, such as Skjold et al 2006 [Skjold A. et al. An Apparent Unidirectional Influx Constant for Manganese as a Measure of Myocardial Calcium Channel Activity; Journal of Magnetic Resonance Imaging; 24:1047-1055, 2006], require multiple measurements of T1 in heart tissue over time to allow for construction of the data set needed for the linear regression to estimate the uptake rate constant. In particular, multiple images for a single time interval are required to find T1 blood measurement at said time interval. Typically, around 11 images are needed to find a single T1 blood measurement at a single time interval. This limits the prior art methods using MRI to a single tissue image cross section and may potentially overlook pathologies in the heart in regions not covered by the selected view. The methods also require absolute and accurate measurements of T1 in myocardial tissue and blood in a period with rapid changes of the manganese concentration. T1 mapping techniques require breath hold and measurement of data from around 11 heart beats and lasts typically around 15 seconds. T1 measurements in the myocardium and blood with the T1 mapping method during infusion of a contrast agent are therefore often inaccurate.

To convert the relaxation rate of blood to the relaxation rate of plasma, the following equation can be used:

$$R {1}_{P L A S M A} = \frac{R {1}_{blood}}{( 1 - h c t )} \qquad ( 7 )$$

### SUMMARY OF THE INVENTION

According to a first aspect of the invention there is provided a method for determining a cardiomyocyte manganese ion uptake rate constant in one or more regions of interest in a heart of a subject comprising: a providing step comprising: providing a left ventricular myocardial image slice covering one or more regions of interest taken during a first time period; providing one or more longitudinal relaxation time weighted images of the left ventricular blood pool taken during the first time period to measure an average signal intensity in blood during the first time period; providing a hematocrit measurement; providing an extracellular volume measurement for each of the one or more regions of interest; an administration step comprising administering a contrast agent comprising manganese 2+ ions; an image acquisition step comprising: performing image acquisition of the left ventricular blood pool using a longitudinal relaxation time weighted pulse sequence at a plurality of time intervals during a second time period to measure respective signal intensities in blood at the plurality of time intervals; performing a longitudinal relaxation time map image acquisition of a left ventricular myocardial image slice covering the one or more regions of interest during a third time period; a measurement step comprising measuring a signal intensity in blood at each of the plurality of time intervals using an image taken at a time point within said corresponding time interval; and a calculation step comprising: calculating a change in a myocardial longitudinal relaxation rate for each region of interest from a baseline myocardial longitudinal relaxation rate extracted from the myocardial image slice taken during the first time period and a myocardial longitudinal relaxation rate at an end time point of an MRI examination extracted from the myocardial image slice during the third time period; calculating a blood longitudinal relaxation rate for each of the plurality of time intervals using respective signal intensities in blood at the plurality of time intervals and a relationship between signal intensity and said plurality of relaxation rates according to the signal intensity equation:

$$SI ∝ \frac{sin α ( 1 - {e}^{- TD * R 1} )}{1 - {e}^{- TD * R 1} cos α} ;$$

wherein α is a flip angle, TD is a trigger delay, SI is the signal intensity, and R1 is the blood longitudinal relaxation rate; calculating a change in blood longitudinal relaxation rate for each of the plurality of time intervals and for the MRI examination end timepoint; integrating over all of the changes in blood longitudinal relaxation rate for each of the plurality of time intervals; and using the integral over all the changes in blood longitudinal relaxation rate in conjunction with the myocardial longitudinal relaxation rate at the end time point in a proportionality relationship with a cardiomyocyte Mn²⁺ uptake rate constant to estimate the cardiomyocyte Mn²⁺ uptake rate constant for each of the one or more regions of interest; wherein said first time period is before an administration of the contrast agent, said second time period is during and after an administration of the contrast agent, and said third time period encompasses the end timepoint of an MRI examination.

Performing image acquisition of the left ventricular blood pool may consist of taking a single image per time interval.

Calculating the change in blood longitudinal relaxation rate for each of the plurality of time intervals may be achieved by: providing, during the providing step, the longitudinal relaxation time weighted map of the left ventricular blood pool taken during the first time period in the form of a saturation recovery imaging pulse sequence taken during the first time period; performing, during the image acquisition step, image acquisition of the left ventricular blood pool using the longitudinal relaxation time weighted pulse sequence at a plurality of time intervals in the form of a saturation recovery pulse sequence at the plurality of time intervals during a second time period; calculating a saturation recovery calibration constant from the saturation recovery imaging pulse sequence taken during the first time period and the average signal intensity in the blood; and using the saturation recovery calibration constant and the signal intensities in blood at the plurality of time intervals to calculate the blood longitudinal relaxation rate during the second time interval.

The method may further comprise using the estimated cardiomyocyte Mn²⁺ uptake rate constant for each region of interest to indicate a T-tubule density at said corresponding region of interest.

The one or more regions of interest may comprise a plurality of regions of interest across a left ventricular myocardium of the heart, and wherein the estimated cardiomyocyte Mn²⁺ uptake rate constant for each of the plurality of regions of interest provides a t-tubule density estimate map across said left ventricular myocardium.

The hematocrit, the ECV in the at least one region of interest and the integral over all the changes in blood relaxation for each of the plurality of time intervals may be used in the following equation:

$${K}_{Cell}^{Mn} = \frac{Δ R {1}_{ROI} - ECV * \frac{Δ R {1}_{blood}}{( 1 - hct )}}{( 1 - ECV ) * \frac{{\int }_{0}^{{t}_{E}} Δ R {1}_{blood} ( t ) dt}{( 1 - hct )}}$$

wherein ΔR1_{ROI} is the change in myocardial longitudinal relaxation rate in the region of interest, ΔR1_{blood} is the change in blood longitudinal relaxation rate, ECV is the extracellular volume fraction for the region of interest. hct is the hematocrit, and

$${\int }_{0}^{{t}_{E}} Δ R {1}_{blood} ( t ) d t$$

is the integral over the change in blood longitudinal relaxation rate from the second time period to the third time period; to estimate of the cardiomyocyte Mn²⁺ uptake rate constant.

Providing at least one left ventricular image slice in the form of a longitudinal relaxation time map taken during a first time period, and/or performing acquisition of myocardial image slices using a longitudinal relaxation time map may comprise using a modified look-locker inversion recovery longitudinal relaxation time map.

Each image in the saturation recovery imaging pulse sequence to measure the average signal intensity in the blood may be obtained using: a 90 degree flip angle; an echo time less than 1 millisecond; and a trigger delay in the range of 150 to 1000 milliseconds.

The contrast agent comprising manganese 2+ ions may have a log stability constant of 10 to 18.

According to a second aspect of the invention there is provided a computer readable media comprising instructions that, when executed on a processor, performs the tasks of: determining a cardiomyocyte manganese 2+ ion uptake rate constant in one or more regions of interest in a heart of a subject by: a) retrieving a myocardial image slice covering the one or more regions of interest at a first time period to retrieve a baseline myocardial longitudinal relaxation rate; b) retrieving a myocardial image slice covering the one or more regions of interest at a third time period to retrieve an examination end time myocardial longitudinal relaxation rate; and c) calculating a change in a myocardial longitudinal relaxation rate at each region of interest using the baseline myocardial longitudinal relaxation rate and the examination end time myocardial longitudinal relaxation rate at respective regions of interest; and d) retrieving a longitudinal relaxation time map of left ventricle blood pool from the first time period to measure a baseline longitudinal relaxation time in blood; e) retrieving an average signal intensity in the blood from the first time period; and f) retrieving a signal intensity in blood at a plurality of time intervals during a second time period; and g) measuring a signal intensity in blood at each of the plurality of time intervals using an image taken at a respective time interval; h) calculating a blood longitudinal relaxation rate for each of the plurality of time intervals using the retrieved signal intensity in blood at the corresponding plurality of time intervals, and a relationship between signal intensity and said plurality of relaxation rates according to the equation:

$$SI ∝ \frac{sin α ( 1 - {e}^{- TD * R 1} )}{1 - {e}^{- TD * R 1} cos α} ;$$

wherein α is a flip angle, TD is a trigger delay, SI is the signal intensity, and R1 is the blood longitudinal relaxation rate; and i) retrieving a blood longitudinal relaxation rate at the first time period; and j) calculating a change in blood longitudinal relaxation rate for each of the plurality of time intervals using the blood longitudinal relaxation rate for each of the plurality of time intervals and the blood longitudinal relaxation rate at the first time period; k) integrating over the change in blood longitudinal relaxation rate for all time intervals; and l) retrieving a hematocrit value; m) retrieving an extracellular volume for the region of interest; and n) calculating the manganese 2+ ion cellular uptake rate constant using a proportionality relationship between: the change in myocardial longitudinal relaxation rate at the region of interest; the change in blood longitudinal relaxation rate; the hematocrit; the extracellular volume at the region of interest; and the integral of the change in blood longitudinal relaxation rate for all time intervals; wherein the first time period is before an administration of a contrast agent comprising manganese 2+ ions, the second time period is during and after the administration of the contrast agent, and the third time period encompasses an end time in the examination.

According to a third aspect of the invention there is provided a system for determining a cardiomyocyte manganese 2+ ion uptake rate constant in one or more regions of interest in a heart of a subject comprising: a database module; a measuring module; a calculation module; and the computer readable media of the second aspect; wherein the computer readable media of the second aspect is distributed across at least the measuring module and the calculation module of the system; wherein the computer readable media distributed in the measuring module is configured, when executed on a processor to perform at least task g); and wherein the computer readable media distributed in the calculation module is configured, when executed on a processor to perform at least task h), j) and n).

Calculating a blood longitudinal relaxation rate for each of the plurality of time intervals may comprise: calculating a saturation recovery calibration constant at the region of interest using the retrieved longitudinal relaxation time map of the left ventricle blood from the first time period and the average signal intensity in the blood at the first time period; and calculating a blood longitudinal relaxation rate for each of the plurality of time intervals using the retrieved signal intensity in blood at the corresponding plurality of time intervals, and the saturation recovery calibration constant; wherein the average signal intensity was retrieved from a saturation recovery imaging pulse sequence; and wherein retrieving the signal intensity in blood at the plurality of time intervals during the second time period comprises: accessing a pre-performed left ventricular imaging slice covering the left ventricular blood pool using a Saturation Recovery pulse sequence at the plurality of time intervals; or performing image acquisition of the left ventricular blood pool using a saturation recovery pulse sequence at a plurality of time intervals during a second time period to measure respective signal intensities in blood at the plurality of times intervals.

Retrieving a myocardial image slice at the region of interest at the third time period may comprise: accessing a pre-performed myocardial image slice taken during the third time period; or performing a longitudinal relaxation map covering all of the one or more regions of interest during the third time period.

Calculating a change in a myocardial longitudinal relaxation rate at the region of interest further may comprise using the equation:

$$Δ R {1}_{ROI} ( {t}_{E} ) = \frac{1}{T {1}_{ROI} ( {t}_{E} )} - \frac{1}{T {1}_{ROI} ( {t}_{B L} )}$$

wherein ΔR1_{ROI}(t_{E}) is the change in myocardial longitudinal relaxation rate at the region of interest at the end time of the MRI examination, T1_{ROI}(t_{E}) is the longitudinal relaxation time at the region of interest taken at the examination end time, and T1_{ROI}(t_{BL}) is the longitudinal relaxation time at the region of interest taken during the first time period.

Calculating a saturation recovery calibration constant at the region of interest may further comprise using the equation:

$${k}_{S R} = \frac{{SI}_{b l o o d} ( {t}_{B L} )}{( 1 - {e}^{- T D / T {1}_{b l ood} ( {t}_{B L} )} )}$$

wherein SI_{blood}(t_{BL}) is the average signal intensity in blood at the first time period, TD is the trigger delay, and T1_{blood}(t_{BL}) is the longitudinal relaxation time in blood taken during the first time period.

Calculating a blood longitudinal relaxation rate for each of the plurality of time intervals may further comprise using the equation:

$$R {1}_{blood} ( t ) = \frac{- ln ( 1 - \frac{{SI}_{blood} ( t )}{{k}_{S R}} )}{T D}$$

wherein SI_{blood}(t) is a signal intensity in blood at the plurality of time intervals, k_{SR} is the saturation recovery calibration constant, and TD is trigger delay.

Calculating a change in blood longitudinal relaxation rate for each of the plurality of time intervals may comprise using the equation:

$$Δ R {1}_{blood} ( {t}_{i} ) = R {1}_{blood} ( {t}_{i} ) - R {1}_{blood} ( {t}_{B L} )$$

wherein R1_{blood}(tᵢ) is the blood longitudinal relaxation rate for each of the plurality of time intervals tᵢ and R1_{blood}(t_{BL}) is blood longitudinal relaxation rate at the first time period.

Calculating the manganese ion cellular uptake rate constant may comprise using the equation:

$${K}_{Cell}^{Mn} = \frac{Δ R {1}_{ROI} - ECV * \frac{Δ R {1}_{blood}}{( 1 - hct )}}{( 1 - ECV ) * \frac{{\int }_{0}^{{t}_{E}} Δ R {1}_{blood} ( t ) dt}{( 1 - hct )}}$$

wherein ΔR1_{ROI} is the change in myocardial longitudinal relaxation rate in the region of interest, ΔR1_{blood} is the change in blood longitudinal relaxation rate, ECV is the extracellular volume for the region of interest and hct is the hematocrit.

According to a fourth aspect of the invention there is provided a use of a contrast agent containing manganese 2+ ions in the method of any of claims 1 to 10 to determine a cardiomyocyte manganese ion uptake rate constant in more or more regions of interest in a heart of a subject.

### BRIEF DESCRIPTION OF THE DRAWINGS

FIG. 1 is a diagram of a flux of calcium ions and manganese ions in a ventricular cardiomyocyte;

FIG. 2a shows a flowchart of instruction steps to be carried out by computer readable media associated with a system of the invention;

FIG. 2b shows a system according to the invention;

FIG. 3 shows a plurality of diagrammatic sections of the heart;

FIG. 4 shows a flowchart of a method of the invention;

FIG. 5 is a graphical representation of a change in blood relaxation rate during and after administration of manganese dipyridoxyl diphosphate;

FIG. 6a shows a graph wherein the gradient provided is a cardiomyocyte manganese ion uptake rate constant as provided by a prior art method;

FIG. 6b shows a graphical representation of correlation between results of the method of the invention herein versus results of a previous method;

FIG. 7 shows a graphical representation of a ratio between changes in relaxation rate between myocardium and plasma after administration of gadolinium dodecane tetraacetic acid;

FIG. 8a shows a graphical representation of a change in longitudinal relaxation rate in the myocardium in a human subject and in a pig subject during and after administration of manganese dipyridoxyl diphosphate; and

FIG. 8b shows a graphical representation of a change in a blood relaxation rate in a human subject and in a pig subject during and after administration of manganese dipyridoxyl diphosphate.

### DEFINITIONS

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

In this text, the term ‘subject’ means any human or non-human animal, and encompasses, and may be limited to, “patient”.

In this text, the term ‘Mn²⁺’ refers to the manganese ion with a positive charge of +2. It is the cationic form of the element manganese (Mn) when it loses two electrons.

In this text, ‘MnDPDP’ refers to manganese dipyridoxyl diphosphate and may be used as a contrast agent containing manganese 2+ ions.

In this text, ‘ZnDPDP’ refers to Zinc dipyridoxyl diphosphate and results in the blood stream when Zinc 2+ ions replace Manganese 2+ ions in MnDPDP′ in Zinc-transmetallation.

In this text, ‘Gd-DOTA’ refers to gadolinium dodecane tetraacetic acid and may also be used as a contrast agent containing gadolinium 3+ ions.

### DETAILED DESCRIPTION

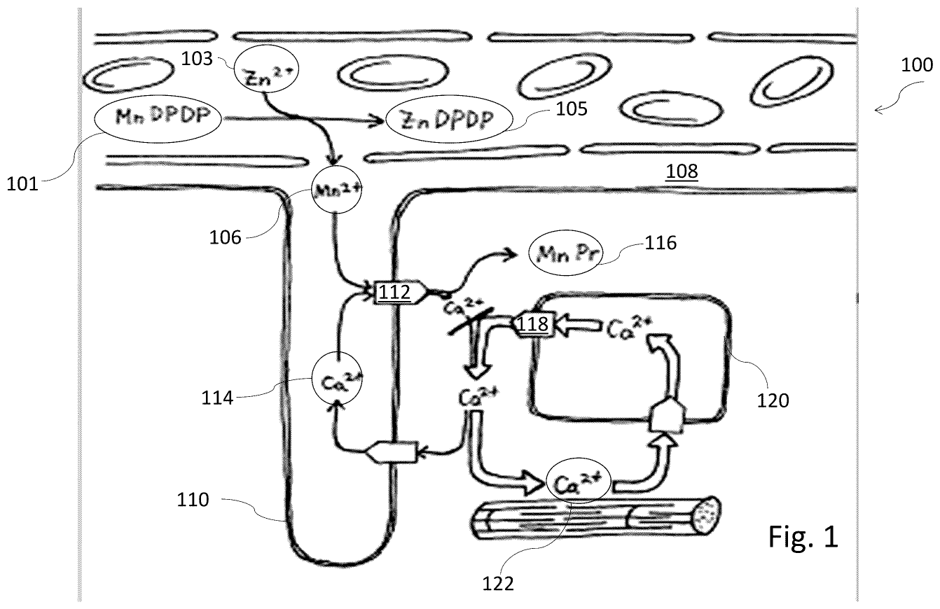

With further reference to FIG. 1, in an Mn²⁺-based MRI contrast agent MnDPDP 101 with a stability constant for Mn²⁺ between those for Ca²⁺ and Zn²⁺ 103, the Mn²⁺ ion 106 will be released by Zinc-transmetallation ZnDPDP 105, and will rapidly diffuse into the extracellular space 108 and T-tubuli 110 where it will be transported into cardiomyocytes, through voltage-gated LTCCs 112 in the period the channels are open after each action potential. The influx rate constant for Mn²⁺ is proportional to the influx of calcium through the same LTCCs 112. The LTCCs 112 are normally in close contact with Ryanodine Receptors (RyR) 118 located in the sarcoplasmic reticulum membrane 120 in a structure called a dyad. The RyRs 118 act as an amplifier of the calcium current through the LTCCs 112 leading to a rapid and spatially synchronized increase in intracellular cytoplasmic 122 calcium which is necessary for effective cardiomyocyte contraction.

Many heart diseases affect the density of the T-tubuli and the integrity of the cardiac dyads. Loss of T-tubuli and the following reduction in the number of LTCCs may lead to a reduced influx rate of calcium, a slow release of calcium from the RyRs and slow increase in intracellular calcium leading to ineffective contraction and a risk of arrhythmias. Concurrently, manganese uptake will be reduced as a consequence of the reduced T-tubule density. Therefore, a method for the assessment of the Mn²⁺ uptake rate can quantify the influx rate of calcium and the T-tubule density and may be of significant clinical value. The T-tubule density varies between regions in the normal heart and heart diseases may affect only parts of the left ventricular wall. Therefore, a method to estimate variations in T-tubule density to be applicable to all segments of the left ventricular wall, not only to a single 2D slice, is desired.

Previously, the proposed methods for estimating a cardiomyocyte manganese ion uptake rate constant, for example those proposed by Patlak and Skjold as referred to above, and a method for estimating a cardiomyocyte manganese ion uptake rate constant further improved by Østensen, require multiple measurements per time interval of T1 in heart tissue over time to allow for construction of a data set needed for a linear regression to estimate the uptake rate constant. In particular, multiple images for a single time interval are required to find T1 blood measurement at said time interval. Typically, around 11 images are needed to find a single T1 blood measurement at a single time interval. This limits the prior art methods to a single tissue image cross section and may potentially overlook pathologies in the heart in regions not covered by the selected view. The methods also require measurements of absolute T1 value in myocardial tissue in a period with rapid changes of the manganese concentration. T1 mapping techniques require data from a plurality, e.g., 11, heart beats and lasts typically around 15 seconds. T1 measurement of the myocardium with the T1 mapping method during infusion of contrast agents is therefore often inaccurate.

The invention described herein addresses the above-mentioned problems of the current methods for estimating a cardiomyocyte manganese ion uptake rate constant. In particular, the invention provides a method which avoids the requirement for taking multiple images of the heart per time interval during contrast agent administration and thus avoids inaccuracies in the resulting calculated constant due to contrast agent concentration changes over elapsed time. The method, computer readable media and the system herein further avoid use of a linear regression method to derive the constant and uses improved technology to acquire different parameters to calculate the constant cardiomyocyte manganese ion uptake rate constant which, in turn, provides an indication for T-tubules density.

With further reference to FIG. 1, a contrast agent MnDPDP 101 which releases Mn²⁺ ions 106 is used. Due to the small size of the Mn²⁺ ions 106, they diffuse rapidly into the ECF 108 and into the T-tubuli 110 where the voltage gated LTCCs 112 are located. Each time an action potential leads to opening of the LTCC 112, Mn²⁺ 106 is transported into the cell in proportion to its extracellular concentration. Mn²⁺ which is inside the cell, binds to proteins to form manganese proteins (MnPr) 116 and is irreversibly trapped into the cell for a time span corresponding to a duration of an MRI scan. Therefore, the increase in the amount of intracellular MnPr 116 is proportional to the integral of the increase in extracellular concentration caused by injection of the contrast agent.

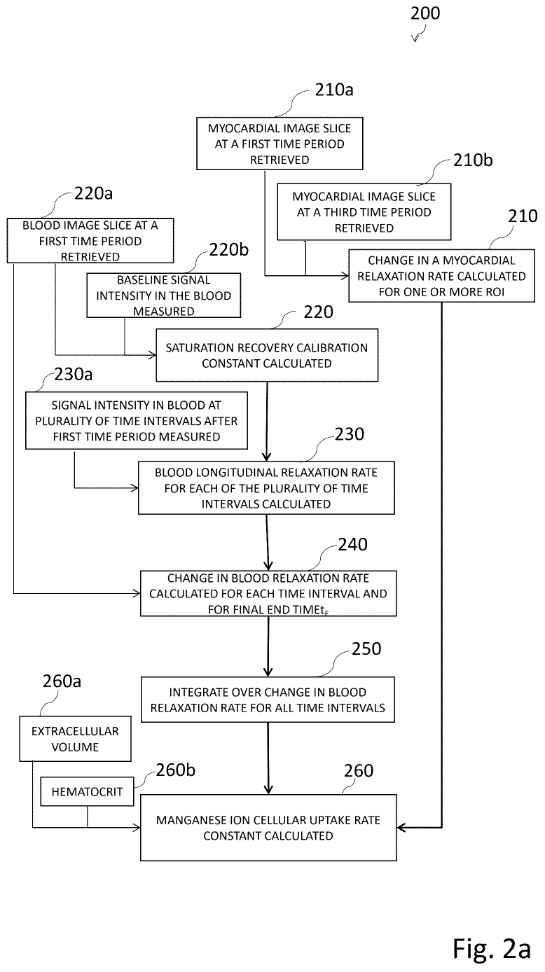

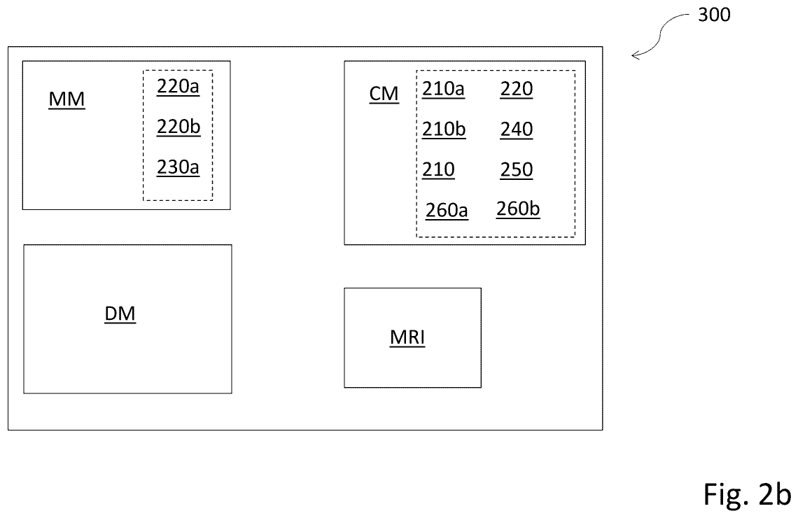

In a first aspect of the invention there is provided computer readable media having stored thereon instructions that, when executed on a processor performs the task of determining a cardiomyocyte manganese ion uptake rate constant, and thereby additionally indicating T-tubule density of a corresponding tissue volume. The tasks performed by the instructions, when executed on a processor, stored on the computer readable media are shown by the steps in FIG. 2a.

With reference to FIG. 2a, at step 210, a change in a myocardial relaxation rate at one or more regions of interest (ROI) is calculated. This is achieved by retrieving one or more myocardial image slices covering each of the one or more ROI taken during the first time period, step 210a. One or more myocardial image slices covering each of the one or more ROI are retrieved, said image slices taken during a third time period, step 210b. The first time period is before an administration of a contrast agent comprising manganese (Mn²⁺) ions, such as MnDPDP. The third time period is after the administration of the contrast agent comprising manganese (Mn²⁺) ions. In particular, for calculation of the change in a myocardial relaxation rate at each ROI, the image slice taken at the third time period is taken at an end time of an MRI examination (the). The change in a myocardial relaxation rate at each ROI is then calculated using the one or more myocardial image slices at each ROI taken during the first time period and the end time of the MRI examination. The one or more myocardial image slices at each ROI for the first time period is retrieved from a storage location in which it was stored after it was pre-performed. The one or more myocardial image slices at each ROI at the may have also been pre-performed and, thus, simply retrieved from a storage location in which it was stored after it was pre-performed. Alternatively, the system may further comprise an MRI scanner and the system may be configured to perform the acquisition of the one or more myocardial images at t_{E}.

Preferably, the one or more myocardial image slices for both the first time period and the time the are longitudinal relaxation time maps (T1 maps). More preferably the one or more myocardial image slices are Modified Look-Locker inversion recovery (MOLLI) T1 maps.

The following is an illustrative, non-limiting MOLLI regime. Two to three inversion pulses followed by several single-shot b-SSFP readouts at various fixed inversion times may be used. Short rest periods may be interspersed to allow recovery of longitudinal magnetization between cycles. In 5(3)3 MOLLI, measurements are obtained at different inversion times over 5+3=8 heart beats with a 3-beat recovery period in between. The inversion pulses are placed so that readout periods all occur at the same place in the cardiac cycle, allowing pixel-wise T1 calculations to be performed.

In particular, the change in a myocardial relaxation rate at the region of interest is calculated using the formula:

$$Δ R {1}_{ROI} ( {t}_{E} ) = \frac{1}{T {1}_{ROI} ( {t}_{E} )} - \frac{1}{T {1}_{ROI} ( {t}_{B L} )} \qquad ( 8 )$$

wherein ΔR1_{ROI}(t_{E}) is the change in myocardial relaxation rate at a region of interest at the end time of the MRI examination, T1_{ROI}(t_{E}) is a myocardial image slice covering a region of interest taken at the examination end time, and T1_{ROI}(t_{BL}) is a myocardial image slice covering a region of interest taken at a baseline timepoint during the first time period.

At step 220, a saturation recovery calibration constant for a ROI in the blood is calculated.

In particular, the saturation recovery calibration constant at the region of interest in blood can be calculated using the equation:

$${k}_{S R} = \frac{{SI}_{b l o o d} ( {t}_{B L} )}{( 1 - {e}^{- T D / T {1}_{b l ood} ( {t}_{BL} )} )} \qquad ( 9 )$$

wherein TD is a trigger delay, T1_{blood}(t_{BL}) is a baseline longitudinal relaxation time in blood, and SI_{blood}(t_{BL}) is a baseline signal intensity in blood, i.e., a signal intensity of the blood during the first time period.

Terms required for calculation of the saturation recovery calibration constant at the ROI in blood are retrieved as follows.

A longitudinal relaxation time map of left ventricle blood from the first time period is retrieved 220a and T1_{blood}(t_{BL}) is measured accordingly.

Since it is the heart organ which is imaged, blood and myocardium can be imaged in the same image slice. Thus, the longitudinal relaxation time map of left ventricle blood pool can be provided by the previous myocardial image slice covering each of the one or more ROI taken during the first time period.

A value for the baseline signal intensity of the blood (SI_{blood}(t_{BL})) and may be retrieved 220b directly from a pre-measured value stored in accessible storage media.

For the SI_{blood}(t_{BL}), pre-administration (first time period) saturation recovery signals may be retrieved and use these to measure SI_{blood}(t_{BL}).

A baseline longitudinal relaxation rate (R1_{blood}(t_{BL})) can also be calculated using the measured T1_{blood}(t_{BL}) by the relationship:

$$R {1}_{blood} ( {t}_{B L} ) = \frac{1}{T {1}_{blood} ( {t}_{B L} )} \qquad ( 10 )$$

Equation (9) can be derived starting with a general signal equation for an MRI signal intensity (SI) from a region with a relaxation rate R1 with a flip angle of a degrees:

$$SI ∝ \frac{sin α ( 1 - {e}^{- T D * R 1} )}{1 - {e}^{- T D * R 1} cos α} {e}^{- T E * R 2} \qquad ( 11 )$$

By using a very short echo time, for example TE=0.8 ms, the R2* effect is negligible e^{-TE*R2*}≈1, leading to equation 11a.

$$SI ∝ \frac{sin α ( 1 - {e}^{- T D * R 1} )}{1 - {e}^{- T D * R 1} cos α} {e}^{- T E * R 2} \qquad ( 11 a )$$

By further using a 90 degree flip angle for which sin(90)=1 and cos(90)=0, the signal intensity becomes proportional to (1−e^{-TD*R1}) with a proportionality constant k_{SR}:

$$SI = {k}_{S R} * ( 1 - {e}^{- T D * R 1} ) \qquad ( 12 )$$

k_{SR} is then derived by rearranging equation (12) to arrive at equation (9).

In an alternative wherein general T1 weighted images of a left ventricular blood pool are used, in place of saturation recovery signals, the saturation recovery calibration constant is not used.

After performing step 220, step 230 is performed. In step 230, a blood longitudinal relaxation rate for each of a plurality of time intervals (tᵢ) is calculated. This is achieved by retrieving a single left ventricular blood pool image at a first time interval in the plurality of time intervals during the second time period. Then using the single image at the first time interval, measuring a signal intensity in blood at the first time interval. a single left ventricular blood pool image is retrieved at a second time interval in the plurality of time intervals during the second time period. Then the single image at the second time interval is used to measure a signal intensity in blood at the second time interval. This process is repeated for all of the time intervals ti in order to measure signal intensity in the blood at each of the plurality of time intervals during the second time period 230a, wherein each of the signal intensity in the blood at each of the plurality of time intervals is measured from a single left ventricular blood pool image at the respective time interval. Then, using the signal intensity in blood at the first time interval along with the saturation recovery calibration constant calculated in step 220, the blood longitudinal relaxation rate for of the first time intervals is calculated. Using the signal intensity in blood at the second time interval along with the saturation recovery calibration constant calculated in step 220, the blood longitudinal relaxation rate for of the second time intervals is calculated. This process is repeated for all of the time intervals ti in order to calculate the blood longitudinal relaxation rate for each of the plurality of time intervals in the second time period. Preferably, the time intervals are synchronized with respiration. The time intervals for the purpose of finding the blood longitudinal relaxation rate occur during the second time period during and after administration of the contrast agent containing manganese (Mn²⁺) ions, before the examination end time the (i.e., before the third time period). For example, during MnDPDP infusion, and a first 4 minutes after end of infusion.

In particular, the blood longitudinal relaxation rate for each of a plurality of time intervals is calculated using the equation:

$$R {1}_{blood} ( {t}_{i} ) = \frac{- ln ( 1 - \frac{{SI}_{blood} ( {t}_{i} )}{{k}_{SR}} )}{T D} \qquad ( 13 )$$

wherein SI_{blood}(tᵢ) is a signal intensity in blood at time interval tᵢ, k_{SR} is the saturation recovery calibration constant, and TD is trigger delay.

Wherein general T1 weighted images of a left ventricular blood pool are used, in place of saturation recovery signals, the saturation recovery calibration constant is not used and the blood longitudinal relaxation rate for each of a plurality of time intervals is calculated using the relationship between signal intensity and said plurality of relaxation rates according to equation (11a).

After step 230, a further step 240 is to be executed, wherein a change in blood relaxation rate for each of the plurality of time intervals is calculated. This is achieved by retrieving a baseline blood longitudinal relaxation rate, for example by taking the inverse of the T1_{blood}(t_{BL}) as described above or from extraction from the blood image slice at the first time period 220a, and using this along with the blood longitudinal relaxation rate for each of the plurality of time intervals as calculated in step 230 to calculate the change in the blood longitudinal relaxation rate at each time interval. A change in blood relaxation rate for the examination end time the is also calculated in a similar manner.

In particular, the change in blood relaxation rate for each of the plurality of time intervals is calculated according to the equation:

$$Δ R {1}_{blood} ( {t}_{i} ) = R {1}_{blood} ( {t}_{i} ) - R {1}_{blood} ( {t}_{B L} ) \qquad ( 14 )$$

wherein R1_{blood}(tᵢ) is the blood longitudinal relaxation rate for each of the plurality of time intervals tᵢ and R1_{blood}(t_{BL}) is blood longitudinal relaxation rate at the first time period (i.e. the baseline blood longitudinal relaxation rate).

After step 240, a further step 250 is to be executed, wherein integrating over the change in blood relaxation rate from the time t=0 to the time t=t_{E} is performed. In particular, the below equation is used:

$${\int }_{0}^{{t}_{E}} Δ R {1}_{blood} ( t ) d t$$

wherein ΔR1_{blood}(t) is the change in blood relaxation rate at time t, and t_{E} is the examination end time, time t=0 marks commencement after an end of the first time period i.e., it is the time wherein the contrast agent administration starts. Thus, t=0 is the start of the second time period.

The ΔR1_{blood}(tᵢ) values calculated in step 240 allow the estimation of the integral from time zero to the nth measurement time

$${\int }_{0}^{{t}_{n}} Δ R {1}_{blood} ( t ) dt .$$

The integral may be estimated by applying the trapezoidal method, as shown in equation (15) for all the time intervals from t=0 to t=tₙ.

$${\int }_{{t}_{n - 1}}^{{t}_{n}} Δ R {1}_{blood} ( t ) dt \approx ( {t}_{n} - {t}_{n - 1} ) \frac{Δ R {1}_{blood ( {t}_{n} )} + Δ R {1}_{blood} ( {t}_{n - 1} )}{2} \qquad ( 15 )$$

Another example method to estimate the integral may be the Simpson's rule for equally spaced time intervals tₙ wherein a plurality of time intervals are summed together to provide a total integral from the start of the second time period to the examination end time, as shown in equation (16).

$${\int }_{{t}_{n - 2}}^{{t}_{n}} Δ R {1}_{blood} ( t ) dt \approx \frac{( {t}_{n} - {t}_{n - 2} )}{6} * ( Δ R {1}_{blood} ( {t}_{n - 2} ) + 4 * Δ R {1}_{blood} ( {t}_{n - 1} ) + Δ R {1}_{blood} ( {t}_{n} ) ) \qquad ( 16 )$$

The step 210 can be executed before, after or contemporaneously with steps 220 to 250. However, both steps 210 and steps 220 to 250 must be carried out before step 260.

Next step 260 is to be executed wherein the manganese ion cellular uptake rate constant is calculated. To achieve this a value of the hematocrit is needed as well as a value for the extracellular volume. Thus, in order to calculate the manganese ion cellular uptake rate constant, first a hematocrit and an extracellular volume 260b is retrieved, the hematocrit is homogeneous across the heart.

Preferably, the hematocrit and ECV values are pre-measured and stored in an accessible storage medium.

The value for the ECV in selected ROIs, ECV_{ROI} may be pre-measured according to the following example method. Measuring pre- and 20 minutes post-contrast myocardial and blood T1 after injection of a gadolinium contrast agent and blood hematocrit, performed 1-30 days before or 1-30 days after administration of the contrast agent comprising Mn²⁺. The ECV may then be calculated according to equation (17).

$${ECV}_{ROI} = \frac{\frac{1}{T {1}_{ROI} ( {t}_{post} )} - \frac{1}{T {1}_{ROI} ( {t}_{pre} )}}{\frac{1}{T {1}_{blood} ( {t}_{post} )} - \frac{1}{T {1}_{blood} ( {t}_{pre} )}} * ( 1 - hct ) \qquad ( 17 )$$

The ECV_{ROI} values may alternatively be obtained by any method known to the skilled person. The obtained ECV_{ROI} value is used in the subsequent steps.

After retrieving/calculation of the hematocrit and ECV_{ROI} the manganese ion cellular uptake rate constant is calculated according to the equation:

$${K}_{Cell}^{Mn} = \frac{Δ R {1}_{ROI} ( {t}_{E} ) - {ECV}_{ROI} * \frac{Δ R {1}_{blood} ( {t}_{E} )}{( 1 - hct )}}{( 1 - {ECV}_{ROI} ) * \frac{{\int }_{0}^{{t}_{E}} Δ R {1}_{blood} ( t ) dt}{( 1 - hct )}} \qquad ( 18 )$$

wherein ΔR1_{ROI}(t_{E}) is the change in myocardial relaxation rate in the region of interest, ΔR1_{blood}(t_{E}) is the change in blood relaxation rate from a baseline to a time the, ECV_{ROI} is the extracellular volume for the region of interest. hct is the hematocrit, and

$${\int }_{0}^{{t}_{E}} Δ R {1}_{blood} ( t ) dt$$

is the integral over the change in blood relaxation rate for all time intervals according to step 250.

Since ΔR1 blood is independent of the views of the heart, this method can be applied to multiple slices of the heart and allow measurements of

$${k}_{Mn}^{cell}$$

for all parts of the left ventricle such as the 16 segments used in state-of-the-art cardiac MRI.

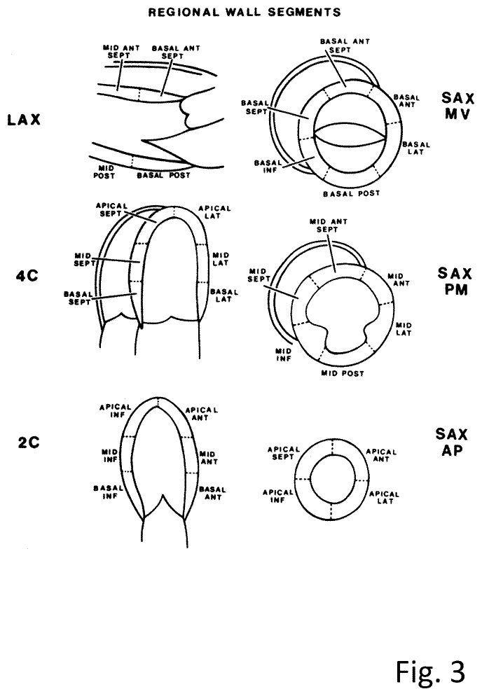

FIG. 3 shows a diagram indicating how a left ventricle myocardium can be divided into 16 segments. These segments consist of a series of longitudinal views (LAX, 4C, 2C) and a series of short-axis views (SAX MV, SAX PM, SAX AP). The longitudinal and short-axis views overlap and complement each other. The longitudinal views comprise a long axis view LAX, a 4-chamber view 4C, a, 2 chamber view 2C. The short-axis views comprise a short-axis mitral valve view SAX MV, a short-axis papillary muscle SAX PM, and short-axis apex SAX AP.

$${k}_{Mn}^{cell}$$

is proportional to influx rate of calcium, LTCC activity and to the local T-tubule density.

FIG. 1 explains why the stability constant for the paramagnetic ion must be lower than for Zn²⁺. If there is no release of free Mn²⁺, there will be no cellular uptake of

$${Mn}^{2 +} and {k}_{Mn}^{cell} = 0 .$$

Solving equation (18), gives

$$Δ R {1}_{ROI} - ECV * \frac{Δ R {1}_{b l o o d}}{( 1 - h c t )} = 0 , and$$

$$ECV = \frac{Δ R {1}_{ROI}}{Δ R {1}_{blood}} * ( 1 - hct ) .$$

This explains why very stable chelates of paramagnetic ions can only be used to measure ECV and do not give information of cellular calcium uptake or T-tubule density. From equation (18) it can also be seen that if ΔR1_{blood}≈0, such as after several halftimes of elimination, the ratio

$$\frac{Δ R {1}_{ROI}}{\int Δ R {1}_{b l o o d}}$$

will indicate the total flux of calcium in a region of interest and the ratio

$$\frac{Δ R {1}_{ROI}}{\int Δ R {1}_{blood}} \frac{( 1 - hct )}{( 1 - {ECV}_{ROI} )}$$

will indicate the T-tubule density in the cells in the ROI.

According to another aspect, there is provided a system for determining a cardiomyocyte manganese 2+ ion uptake rate constant in one or more regions of interest in a heart of a subject comprising: a database module; a measuring module; a calculation module; and the computer readable media of claim 9; wherein the computer readable media of claim 9 is distributed across at least the measuring module and the calculation module of the system.

The measuring module comprises computer readable media having instructions that, when executed on a processor to perform at least the task of measuring a signal intensity in blood at each of the plurality of time intervals using an image taken at a respective time interval. In particular, the measuring model has instructions that, when executed on a processor, perform the task of retrieving a single left ventricular blood pool image at a first time interval in the plurality of time intervals during the second time period. Then using the single image at the first time interval, measuring a signal intensity in blood at the first time interval. a single left ventricular blood pool image is retrieved at a second time interval in the plurality of time intervals during the second time period. Then the single image at the second time interval is used to measure a signal intensity in blood at the second time interval. This process is repeated for all of the time intervals ti in order to measure signal intensity in the blood at each of the plurality of time intervals during the second time period, wherein each of the signal intensity in the blood at each of the plurality of time intervals is measured from a single left ventricular blood pool image at the respective time interval.

The calculation module comprises computer readable media having instructions that, when executed on a processor to perform at least the tasks of calculating a blood longitudinal relaxation rate for each of the plurality of time intervals using the retrieved signal intensity in blood at the corresponding plurality of time intervals, and a relationship between signal intensity and said plurality of relaxation rates according to the equation:

$$SI ∝ \frac{sin α ( 1 - {e}^{- TD * R 1} )}{1 - {e}^{- TD \cdot R 1} cos α} ;$$

wherein α is a flip angle, TD is a trigger delay, SI is the signal intensity, and R1 is the blood longitudinal relaxation rate. The calculation module comprises computer readable media having instructions that, when executed on a processor to perform at least the tasks of calculating a change in blood longitudinal relaxation rate for each of the plurality of time intervals using the blood longitudinal relaxation rate for each of the plurality of time intervals and the blood longitudinal relaxation rate at the first time period. The calculation module comprises computer readable media having instructions that, when executed on a processor to perform at least the tasks calculating the manganese 2+ ion cellular uptake rate constant using a proportionality relationship between: the change in myocardial longitudinal relaxation rate at the region of interest; the change in blood longitudinal relaxation rate; the hematocrit; the extracellular volume at the region of interest; and the integral of the change in blood longitudinal relaxation rate for all time intervals; wherein the first time period is before an administration of a contrast agent comprising manganese 2+ ions, the second time period is during and after the administration of the contrast agent, and the third time period encompasses an end time in the examination.

FIG. 2b shows an example system 300 according to the invention. The system 300 has a measuring module MM, a computational module CM, a database module DM and an optional MRI scanner. The measuring module MM of system 300 has computer readable media comprising the instructions which perform the task 220a, 220b and 230a. The computational module CM of system 300 has computer readable media comprising the instructions which perform the task 210a, 210b, 210, 220, 240, 250 and 260b.

According to a yet further aspect, there is provided a method for determining a cardiomyocyte manganese ion uptake rate constant in at least one region of interest in a heart of a subject.

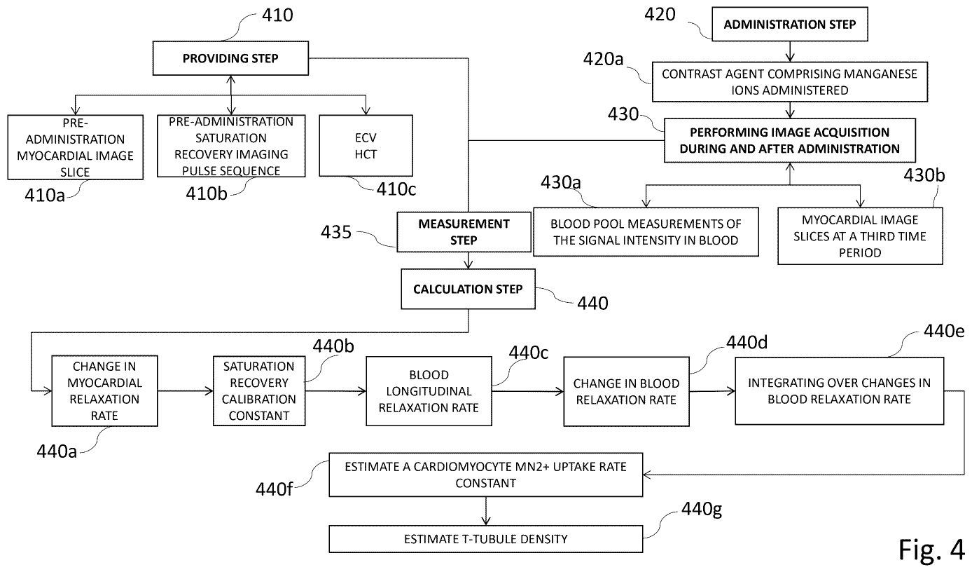

FIG. 4 shows a flowchart of the method 400 for determining a cardiomyocyte manganese ion uptake rate constant in one or more regions of interest in a heart of a subject.

The method 400 has a providing step 410, an administration step 420, a performing image acquisition step 430, a measurement step 435 and a calculation step 440. The providing step 410 can be carried out at any time before, after or contemporaneously with the administration step and/or the performing image acquisition step 430. The providing step 410, the performing image acquisition step 430, and the measurement step 435 are performed before the calculation step 440 such that all necessary information required for the calculation is available. The performing image acquisition step 430 and the measurement step 435 are performed during and after a contrast agent comprising Mn²⁺ ions has been administered into the subject.

The providing step 410 is the providing of images and measurements required to perform the calculation step 440, wherein the images and measurements are either representing a respective ROI before administration of contrast agent, or are independent of the contrast agent and/or ROI. The providing step 410 includes providing at least one left ventricular image slice covering one or more ROIs representing a state before contrast agent administration, the image slice in the form of a longitudinal relaxation time map 410a. In the example wherein a baseline signal intensity is derived from saturation recovery pulse sequence image, the providing step 410 further includes providing a pre-administration saturation recovery pulse sequence image 410b.

Preferably, the saturation recovery imaging pulse sequence is taken with a 90 degree flip angle and a short echo time. With a 90 degree flip angle and a short echo time, equation (11) above can be simplified to equation (12) which itself can be rearranged to give equation (9).

Preferably, the echo time is less than 1 millisecond, and the trigger delay is in the range of 200 to 800 milliseconds, preferably 400 milliseconds.

Alternatively, a baseline signal intensity is derived from general T1 weighted images of a left ventricular blood pool may be used in place of saturation recovery signals. In this case, a blood longitudinal relaxation rate for each of a plurality of time intervals is calculated using the relationship between signal intensity and said plurality of relaxation rates according to equation (11a).

Also provided in the providing step is an ECV associated with each ROI and a hematocrit 410c. The images and measurements in the providing step 410 can be provided at any stage of the method 400 before the calculation step 440, provided that they were taken previously prior to the administration step 420 or when the ROI is unaffected by the contrast agent.

In the administration step 420, a contrast agent comprising manganese ions is administered to the subject 420a. Preferably, a dose DCA μmol kg⁻¹ of a contrast agent at a dosing rate of RCA μmol kg⁻¹ min⁻¹ is administered. Preferably, the contrast agent comprises a Mn²⁺ based contrast agent having a log stability constant of 10 to 18.

The image acquisition step 430 takes place during a second and third time periods. The second time period being during and after the administration of the contrast agent comprising manganese 2+ ions i.e. after the pre-administration first time period. The third time period is after the second time period and encompasses an end timepoint of the examination.

During the image acquisition step 430, image acquisition of blood pool measurements of a signal intensity in blood is performed at a plurality of time intervals 430a during the second time period. For signal intensity, a saturation recovery pulse sequence is preferably used. Yet more preferably, each image in the saturation recovery imaging pulse sequence is taken with a 90 degree flip angle; an echo time less than 1 millisecond; and a trigger delay of 400 milliseconds. Alternatively, general T1 weighted images of a left ventricular blood pool may be used in place of saturation recovery signals. In this case, a blood longitudinal relaxation rate for each of a plurality of time intervals is calculated using the relationship between signal intensity and said plurality of relaxation rates according to equation (11a). Only a single image per time interval is required to measure a signal intensity at that time interval. This is in contrast to prior techniques which measure T1 blood measurement during administration (i.e. during the second time period) for each time interval, in which multiple images per time interval are required. Since multiple images are required to calculate T1 blood measurements for each time interval, the measurement is taken over an elapsed time. Since each time interval occurs in the second time period in which administration occurs, during the elapsed time arising from the need for multiple images per T1 blood (i) measurement per interval, rapid changes in contrast agent concentration can occur. This can lead to inaccuracies in the T1_{blood} (i) measurements. This can ultimately lead to an inaccurate calculation of the cardiomyocyte manganese ion uptake rate constant. In contrast, the present method measures signal intensity for each of the plurality of time intervals. Measurements for signal intensity only require one image per time interval. Capture of a single image does not occur over an elapsed time duration but in a snapshot in time (i.e. at a time point within the time interval), and thus measurements of signal intensity are not effected by inaccuracies of rapid changes in constant agent.

Also performed during the image acquisition step 430 is the acquisition of myocardial image slices using a longitudinal relaxation time map during a third time period, said third time period being after the plurality of time intervals 430b, i.e., an end timepoint of the MRI examination.

The method 400 then progresses to a calculation step 440.

In the calculation step, firstly a change in myocardial relaxation rate at each ROI is calculated 440a from a baseline myocardial longitudinal relaxation rate extracted from the myocardial image slice taken during the first time period and a myocardial longitudinal relaxation rate at an end time point of an MRI examination extracted from the myocardial image slice during the third time interval for each ROI. Step 440a may be achieved using equation (8).

Then, a saturation recovery calibration constant is calculated 440b from a baseline longitudinal relaxation time in blood extracted from the image of the left ventricle blood taken during the first time period, and the average signal intensity in the blood. Step 440b may be achieved using equation (9).

Next a blood longitudinal relaxation rate for each of the plurality of time intervals during the second time interval, using respective signal intensities in blood at the plurality of times intervals, is calculated 440c. Step 440c may be achieved using equation (13). Alternatively, wherein general T1 weighted images of a left ventricular blood pool was used in place of saturation recovery signals, the blood longitudinal relaxation rate for each of a plurality of time intervals is calculated using the relationship between signal intensity and said plurality of relaxation rates according to equation (11a). In this case, step 440b of calculating the saturation recovery calibration constant is not required.

Further, a change in blood relaxation rate for each of the plurality of time intervals is calculated 440d using the blood longitudinal relaxation rate for each of the plurality of time intervals and the baseline blood longitudinal relaxation rate as calculated in the previous steps. A change in blood relaxation rate for the examination end time tE is also calculated in a similar manner. Step 440d may be achieved using equation (14).

Integration is performed over all of the changes in blood relaxation rate for each of the plurality of time intervals 440e.

Finally, a cardiomyocyte Mn²⁺ uptake rate constant is estimated 440f using the integral over all the changes in blood relaxation for each of the plurality of time intervals along with the hematocrit, and the ECV provided in step 410. In particular, the cardiomyocyte Mn²⁺ uptake rate constant is estimated 440f using the equation (18) above.

In particular example to illustrate time frames of the method, for MnDPDP, blood and myocardial T1 values were measured at baseline (i.e. at a timepoint during the first time interval). Saturation recovery was measured during MnDPDP infusion, and the first 4 minutes after end of infusion (for 6 minutes). Then back to T1 mapping every 2 minutes until 30 minutes. For GdDTPA, blood and myocardial T1 values were measured at baseline, and 9 minutes continuously until 10 minutes. Saturation recovery was measured during GdDTPA infusion and the first 7 minutes after end of infusion; final T1 mapping was done at 10 minutes.

The method of the invention does not require multiple T1 images of the myocardium for each time interval during the infusion and early elimination phase when there may be rapid changes in manganese concentration. Myocardial T1 needs only to be measured at the start and end of the MRI examination. Therefore, mapping over time, with one image per time interval, is only needed for the blood pool to measure the integral of the changes in blood relaxation rate, which is independent of the myocardial tissue properties. This method of measuring the uptake rate constant can be done for the whole heart and not only for a single selected section of the heart which is the case for the methods described in prior art. The uptake rate constant in the cell fraction is a measure of the LTCC activity which is proportional to the T-tubule density since the LTCCs are mainly located in the T-tubule membranes.

The method further allows estimation of calcium uptake rates and thereby T-tubule density at a cellular level, step 440g. The method and system of the invention employ MRI in order to measure the cellular manganese uptake rate constant and determines the manganese uptake rate constant

$${K}_{Cell}^{Mn}$$

at the cellular level in multiple sections of the heart as opposed to prior art methods only applicable at a tissue level in a single slice, and provides a novel method for measurement

$${K}_{Cell}^{Mn} .$$

The methods and uses of the invention are based on cellular uptake of metal ions via LTCCs, measuring the inward flux of the metal ions through the LTCCs using a medical imaging method such as MRI using a chelate of Mn²⁺. Alternatively, the medical imaging method is positron emission imaging (PET) using ^{52g}Mn, ^{52m}Mn or ⁵⁴Mn.

The system and method may be useful for various tissues and organs that comprise cells having LTCCs. In particular, the methods and uses of the invention are useful for the heart, in particular the myocardium.

The system and method allow for determining a cellular Mn²⁺ uptake rate constant of a subject using MRI. Thus, estimates of calcium influx rates and T-tubule densities at a cellular level in one or several regions of interest in the heart can be determined, and is useful for assessment of cardiac function, risk for arrhythmias and for diagnosis and management of heart diseases.

The cardiomyocyte manganese uptake rate and the extracellular volume fraction are measured and used to determine the manganese uptake rate constant at the cellular level as opposed to only at a tissue level. The method therefore allows a separation of variations caused by cellular activity from variations caused by cell numbers and/or partial volume effects. It is the uptake capacity and—importantly—the T-tubule density and LTCC activity that is measured rather than only the total myocardial manganese uptake.

The system and method of the invention also enable the measurement of the cardiomyocyte manganese uptake rate and the extracellular volume fraction for more than one imaging cross section of the heart, optionally a 3D data set for the whole heart as opposed to methods limited to one cross section of the heart.

### EXAMPLES

The following examples demonstrate the success of the above-described system and method in pigs and humans. Pigs were chosen as a test animal because they are thought to have similar cardiac physiology as humans and are large enough to allow MRI in a clinical 3T scanner.

### Example 1: Measurement of the Manganese Uptake Rate Constant in a Pig A Receiving a 2 Minutes Infusion of Mangafodipir

The pig A was premedicated with ketamine, 20 mg kg⁻¹ i.m., azaperone 3 mg kg⁻¹ i.m. and atropine 0.02 mg kg⁻¹ i.m. Induction and maintenance of anaesthesia was done with pentobarbital 50 mg kg⁻¹ i.v. followed by 2 mg kg⁻¹, morphine 1 mg kg⁻¹, lidocaine and incision for tracheostomy tube ventilation, sevoflurane 0.5 MAC and morphine 0.2-3 mg kg⁻¹ hour⁻¹. Ventilation was performed with a respirator at a rate of 20 breaths per minute with a 33% duty cycle (% duration of inspiration). Four lead electrocardiogram (ECG) electrodes were place proximal on each limb for monitoring ECG and triggering the MRI scanner. A catheter was placed in the left carotid artery for blood pressure monitoring and arterial blood sampling.

Hematocrit was measured to 0.257 at the beginning of the experiment. Using a MOLLI with 5(3)3 protocol, T1 in blood was measured to 1641 ms and T1 in septum was 1124 ms. Using a Saturation Recovery (SatRec) protocol before administration of contrast agent, the average Signal Intensity in blood was 7.331 for 50 baseline images. The Saturation Recovery calibration constant k_{SR} was calculated according to equation (9) with a trigger delay TD of 400 ms.:

Substituting the above values into equation (9) gives:

$${k}_{S R} = \frac{7.331}{( 1 - {e}^{- 400 / 1641} )}$$

which gave a value of k_{SR} equal to 33.89.

At time t=0, a dose of 5 μmol kg⁻¹ mangafodipir was infused during 2 minutes under continuous ECG triggered SatRec scanning without breath hold. The SatRec scan continued until t_{E}=30 minutes with 437 SI_{blood} measurements.

Using equation (13), the signal intensities were converted to relaxation rate values and then recalculated to changes in blood relaxation rates from baseline using equation (14).

At the end of the experiment at time 30 minutes, the blood relaxation rate was 0.020 s⁻¹ and the integral of the change in blood relaxation rates was calculated using the trapezoidal formula of equation (15) for integration to be 2.035 s⁻¹ min.

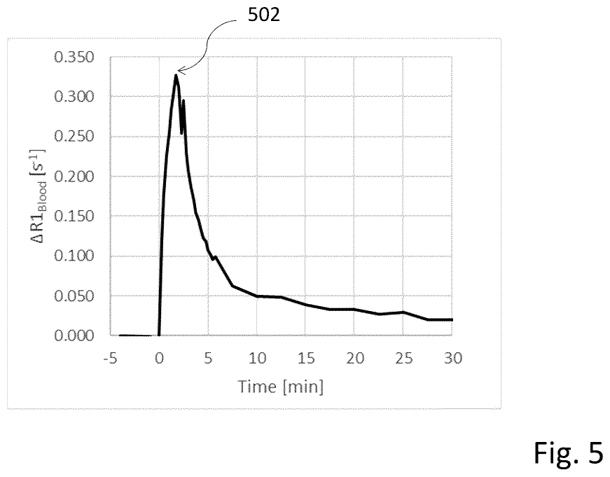

FIG. 5 illustrates change in blood relaxation rate (ΔR1_{blood}) before, during and after infusion of mangafodipir. At time zero, a dose of 5 μmol kg⁻¹ mangafodipir was administered as an infusion of 2.5 μmol kg⁻¹ min⁻¹ for 2 minutes. During infusion there is a rapid increase of blood relaxation rate to a peak 502 increase of 0.328 s⁻¹. When the infusion is stopped there is a rapid reduction of blood relaxation rate. At time 4 minutes, the change in relaxation rate was 0.145 s⁻¹ which is less than half of the peak value, indicating an initial blood halftime of less than two minutes.

At time t_{E}=30 minutes, the T1 in septum was measured to 1064 ms which gave a change in relaxation rate at the end of the experiment of 1000/1064−1000/1124=0.0503 s⁻¹.

Thirty minutes after the manganese experiment, an MRI image was acquired with MOLLI T1 map sequence and septum T1 was measured to 1063 ms and blood T1 was measured to 1618 ms. The pig received a 2 minutes long infusion of 0.050 mmol kg⁻¹ gadoteric acid. Ten minutes after the infusion a MRI image was acquired with MOLLI T1 map sequence and septum was measured to 805 ms and blood T1 was 760 ms. ECV was measured as:

$$ECV = \frac{Δ R {1}_{septum}}{Δ R {1}_{blood}} * ( 1 - hct ) = \frac{\frac{1000}{805} - \frac{1000}{1063}}{\frac{1000}{760} - \frac{1000}{1618}} * ( 1 - 0.257 ) = 0.3 \; 21 ml / ml .$$

Finally, the uptake rate constant for manganese was calculated by substituting the above measured/calculated values into equation (18) to give:

$${K}_{Cell}^{Mn} = \frac{0.0503 - 0.321 * \frac{0.02}{1 - 0.257}}{( 1 - 0.321 ) * \frac{2.035}{1 - 0.257}}$$

which gave a value of

$${K}_{Cell}^{Mn}$$

to 0.0228 min⁻¹.

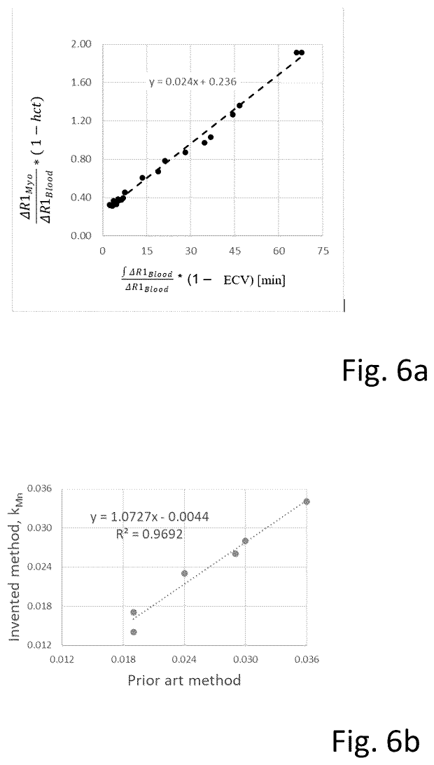

In comparison, the method described in international patent application WO2022/258517A1 (the contents of which are herein incorporated by reference) show a slope of 0.024 min⁻¹, FIG. 6a. The method described herein therefore gave the same estimate of the manganese influx rate constant without having to use linear regression data from measurements of the myocardial relaxation rate during the infusion.

### Example 2: Summary of 6 Pigs Receiving a 2 Minutes Infusion of Mangafodipir

Six domestic pigs with body weights between 50 kg and 60 kg were examined the same way as pig A in Example 1. After baseline measurements of T1 in blood and septum, saturation recovery images were acquired for each heart beat before, during and after a 2 minutes infusion of 5 μmol kg⁻¹ mangafodipir. The baseline blood signal intensity was used to calculate the saturation recovery calibration constant used to calibrate the measurements of change in blood relaxation rate. The integral of the blood time intensity curve was calculated using the trapezoidal rule according to equation (15). At the end of the experiment, after 30 minutes, a T1 map image was acquired and the change in septum relaxation rate was calculated according to equation (8).

Table 1 summarizes the measurements of blood parameters. The average integral of the blood time intensity curves (I_{blood}) were 2.24 s⁻¹ min and the average change in blood relaxation rate at t_{E}=30 minutes was 0.018 s⁻¹.

TABLE 1
Blood parameters in six pigs A-F

| | Time point | | | | | |
|---|---|---|---|---|---|---|
| | First time period | | | | Third time period | |
| | hct | T1_{Blood} | SI_{blood} | k_{SR} | I_{blood} | ΔR1_{blood} |
| Subject # | ml/ml | ms | a.u. | a.u. | s⁻¹ min | s⁻¹ |
| A | 0.257 | 1641 | 7.331 | 34.1 | 2.04 | 0.020 |
| B | 0.237 | 1757 | 5.006 | 24.6 | 2.08 | 0.028 |
| C | 0.266 | 1642 | 8.584 | 39.7 | 2.15 | 0.019 |
| D | 0.261 | 1691 | 6.451 | 30.6 | 2.33 | 0.015 |
| E | 0.273 | 1565 | 5.866 | 26.0 | 2.30 | 0.011 |
| F | 0.244 | 1689 | 6.634 | 31.5 | 2.54 | 0.015 |
| Average | 0.256 | 1664 | 6.645 | 31.1 | 2.24 | 0.018 |

Table 2 summarizes the measurements related to a ROI covering septum for each of the six animals. The average change in myocardial relaxation rate was 0.058 s⁻¹.

TABLE 2
Tissue parameters for six pigs A-F
measured for a ROI covering septum

| | Time point | | | | | | |
|---|---|---|---|---|---|---|---|
| | | | Third time period | | | | |
| | First time period | | | | | ICT- | ICT- |
| | ECV | T1 | T1 | ΔR1 | k_{cell}^{Mn} | slope | icpt |
| Subject # | ml/ml | ms | ms | s⁻¹ | min⁻¹ | min⁻¹ | ml/ml |
| A | 0.321 | 1124 | 1064 | 0.050 | 0.022 | 0.024 | 0.236 |
| B | 0.361 | 1243 | 1141 | 0.072 | 0.034 | 0.036 | 0.207 |
| C | 0.335 | 1171 | 1111 | 0.046 | 0.019 | 0.019 | 0.169 |
| D | 0.329 | 1180 | 1091 | 0.069 | 0.029 | 0.029 | 0.222 |
| E | 0.439 | 1164 | 1099 | 0.051 | 0.019 | 0.019 | 0.413 |
| F | 0.448 | 1161 | 1085 | 0.060 | 0.030 | 0.030 | 0.275 |
| Average | 0.372 | 1174 | 1099 | 0.058 | 0.026 | 0.026 | 0.254 |

The average uptake rate constant for Mn²⁺ was 0.026 min⁻¹. This was not significantly different from the average slope of the regression lines (ICT-slope) measured as described in WO2022/258517A1 and demonstrates that the method described herein, which is not limited to one imaging slice, gives a comparable estimate of the uptake rate constant for Mn²⁺. FIG. 6b shows the correlation of the two methods.

### Example 3: Summary of 2 Pigs Receiving a 7.5 Minutes Infusion of Mangafodipir

Two domestic pigs with body weights 57 kg and 58 kg were examined the same way as pig A in Example 1 with the exception that the infusion of 5 μmol kg⁻¹ mangafodipir was given during 7.5 minutes to test the hypothesis that the estimated manganese uptake rate constant is independent of infusion duration.

Table 3 summarizes the measurements of blood parameters. The average integral (I_{blood}) of the blood time intensity curves was 1.98 s⁻¹ min and the average change in blood relaxation rate at t_{E}=30 minutes was 0.025 s⁻¹.

TABLE 3
Blood parameters in two pigs G, H

| | Time point | | | | | |
|---|---|---|---|---|---|---|
| | First time period | | | | Third time period | |
| | hct | T1_{Blood} | SI_{blood} | k_{SR} | I_{blood} | ΔR1_{blood} |
| Subject # | ml/ml | ms | a.u. | a.u. | s⁻¹ min | s⁻¹ |
| G | 0.238 | 1578 | 6.815 | 30.5 | 2.406 | 0.037 |
| H | 0.271 | 1673 | 7.487 | 35.5 | 1.551 | 0.012 |
| Average | 0.255 | 1626 | 7.151 | 33.0 | 1.978 | 0.025 |

Table 4 below summarizes the measurements related to a ROI covering septum for each of the two animals G, H. The results for 7.5 minutes infusion did not differ from the values for a two-minute infusion and again demonstrates that the method described herein, which is not limited to one imaging slice, gives a comparable estimate of the uptake rate constant for Mn²⁺ as other methods described in the prior art.

TABLE 4
Tissue parameters for two pigs G, H
measured for a ROI covering septum

| | Time point | | | | | | |
|---|---|---|---|---|---|---|---|
| | | | Third time period | | | | |
| | First time period | | | | | ICT- | ICT- |
| | ECV | T1 | T1 | ΔR1 | k_{cell}^{Mn} | slope | icpt |
| Subject # | ml/ml | ms | ms | s⁻¹ | min⁻¹ | min⁻¹ | ml/ml |
| G | 0.452 | 1194 | 1122 | 0.054 | 0.017 | 0.019 | 0.542 |
| H | 0.411 | 1219 | 1133 | 0.062 | 0.038 | 0.037 | 0.189 |
| Average | 0.432 | 1207 | 1128 | 0.058 | 0.028 | 0.028 | 0.366 |

### Example 4: Evidence that ECF is in Equilibrium with Plasma

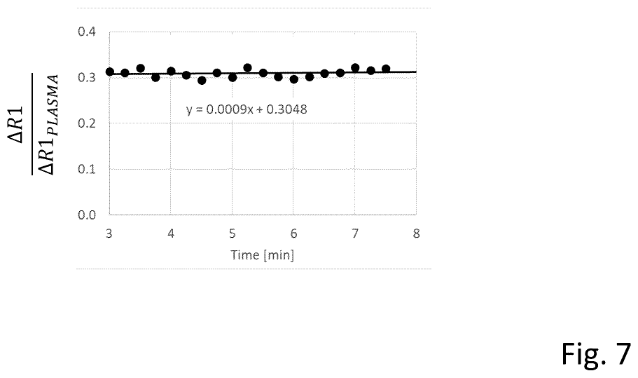

The method of the present invention is based on the assumption that blood plasma and tissue extracellular fluid is near equilibrium after administration of contrast agent according to the invention. This implies that, for a contrast agent that is not taken up by cells, such as gadoteric acid, the ratio between the changes in relaxation rate between myocardium and blood is constant after the end of the infusion. This was found to be the case in all pigs A-H. FIG. 7 shows an example of this in pig A in the study. Changes in plasma were calculated as changes in blood divided by (1−hct). Since the Mn²⁺ ion is smaller and more diffusible than gadoteric acid, this finding provides evidence that the Mn²⁺ concentration in plasma and ECF are substantially equal, or at least significantly similar in the time period, after the infusion.

### Example 5: Comparison of Manganese Uptake Rate in Human and Pig

Pigs represent a species with lower T-tubule density than humans [Heinzel 2002]. Manganese enters cardiomyocytes through LTCCs which are located in the T-tubuli and, thus, the manganese uptake rate constant can be used to estimate the T-tubule density. T1 in blood and the heart septum was measured in a healthy human volunteer and a pig before and 30 minutes after start of infusion of 5 μmol kg⁻¹ mangafodipir. The signal intensity from the blood pool was measured multiple times to allow an estimate of the integral of the change in blood relaxation rate. Hematocrit was measured before the start of the experiment and ECV was measured when the blood relaxation rate was normal and not influenced by manganese.

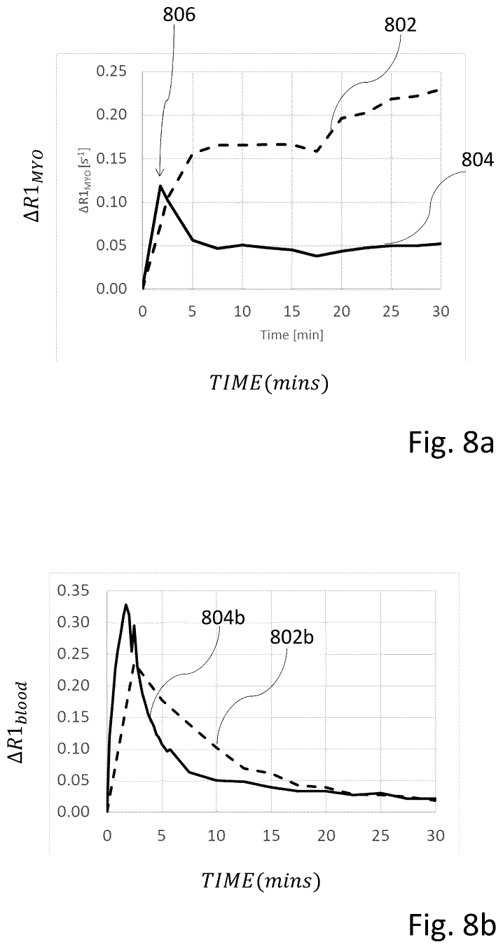

FIG. 8a shows a time intensity curve for the changes in myocardial relaxation rates in the human subject 802 and the pig 804. The relaxation rate in the human myocardium continues to increase after the end of infusion, whereas the relaxation rate in the pig myocardium has a peak 806 at the end of infusion and then declines when the reversible, extracellular signal declines.

Table 5 below summarizes the parameters required to calculate the cardiomyocyte uptake rate constant for manganese in the pig and human according to equation (18).

TABLE 5

| Parameter(t) | Human | Pig |
|---|---|---|
| ΔR1 blood(30)[s⁻¹] | 0.017 | 0.020 |
| ΔR 1 myo(30) [s⁻¹] | 0.229 | 0.052 |
| ∫ΔR 1 blood(30) [s⁻¹min] | 2.37 | 2.11 |
| ECV [ml/ml] | 0.245 | 0.321 |
| hct [ml/ml] | 0.440 | 0.257 |
| k_{cell}^{Mn}[min⁻¹] | 0.0693 | 0.0226 |

The table show that the change in myocardial relaxation rate in the human of 0.229 s⁻¹ is around four times higher than in the pig after 30 minutes. The analysis according to the method described herein show that the supply of manganese from blood represented by the integral of the change in blood relaxation rates is almost the same and the difference in extracellular volume likely only explains a minor part of the difference between pigs and humans. The cellular uptake rate constant.

$${K}_{Cell}^{Mn} ,$$

in the human subject measured according to the method described herein was 0.0693 min⁻¹ which is around three times higher than the value 0.0226 min⁻¹ in the pig subject. This demonstrates a genuinely reduced cellular manganese uptake rate constant in the pig subject, which is presumably caused by the low T-tubule density in the pig species leading to reduced number of L-type calcium channels and reduced capacity to transport manganese and calcium into the cardiomyocytes.

A difference in uptake could be caused by differences in the supply from blood. FIG. 8b show that there was no significant difference in the change in relaxation rate in blood (R1_{blood}) between the human 802b and the pig 804b. The integrals of the blood time intensity curves were also similar, 2.37s⁻¹ min and 2.11 s⁻¹ min, respectively. The cellular uptake rate constants of 0.069 min⁻¹ in the human and 0.023 min⁻¹ in the pig, demonstrating that the method can be used to identify tissue with low t-tubule density. The high t-tubule density in humans implies a higher uptake in late time points when blood signal is low.

The invention may be particularly useful in patients with heart failure, and/or with cardiomyopathies leading to heart failure, and specifically very useful in patients with suspected heart failure with preserved ejection fraction (HFpEF), heart failure with reduced ejection fraction (HFrEF), dilated cardiomyopathy, hypertrophic cardiomyopathy, diabetic cardiomyopathy, hypertensive cardiomyopathy, and/or “broken heart syndrome” i.e. takotsubo cardiomyopathy. Advantageously, the invention provides an improved evaluation for cardiomyocyte calcium handling, aiding the diagnosis and treatment of heart failure. Hence, the subject may have been diagnosed with or is suspected of having heart failure and/or a cardiomyopathy leading to heart failure. Preferably, the subject has been diagnosed with, or is suspected of having, HFpEF, HFrEF, dilated cardiomyopathy, hypertrophic cardiomyopathy, diabetic cardiomyopathy, hypertensive cardiomyopathy, and/or “broken heart syndrome” i.e., takotsubo cardiomyopathy. Even more preferably, the subject has been diagnosed with, or is suspected of having, heart failure with HFpEF.

Specifically, the present invention is very useful in the diagnosis of patients suffering from HFpEF, a form of heart failure in which the ejection fraction—the percentage of the volume of blood ejected from the left ventricle with each heartbeat divided by the volume of blood when the left ventricle is maximally filled—is normal, defined as greater than 50%. Approximately half of heart failure patients have preserved ejection fraction. HFpEF is characterised by abnormal diastolic function: there is an increase in the stiffness of the left ventricle, which causes a decrease in left ventricular relaxation during diastole. In the early stages of HFpEF, the strain on the tissue may lead to a compensatory reaction in the cardiomyocytes leading to increased uptake rate, as seen also in the remote areas after acute myocardial infarction. Thus, the invention enables the identification of increased T-tubule density and calcium flux as a cause of myocardial stiffness. Later in the development of HFpEF, fibrous tissue starts to form leading to increased ECV. The invention may still detect cardiomyocyte hypercontraction as a contributor to stiffness, whereas the conventional method may erroneously report this as a reduction. If myocyte replacement becomes more significant, the conventionally used method will grossly underestimate the cellular function.

Preferred uses include, but are not limited to, the diagnosis of HFpEF, and the assessment of the contribution from cardiomyocytes to the ventricular stiffness. Other preferred uses include the diagnosis of HFrEF, dilated cardiomyopathy, hypertrophic cardiomyopathy, diabetic cardiomyopathy, hypertensive cardiomyopathy, and/or “broken heart syndrome” i.e., takotsubo cardiomyopathy. The invention is further useful for treatment monitoring; with repeated examinations, the development of disease and/or the responses to therapy can be followed.

Having described preferred examples of the invention it will be apparent to those skilled in the art that other embodiments incorporating the invention may be used. The model used for data analysis is based on few parameters and averaging signals from regions of interest and may be further improved by novel techniques using machine learning and artificial intelligence on the huge data sets generated by the MRI examinations. These and other examples of the invention illustrated above are intended by way of example only and the actual scope of the invention is to be determined from the appended claims.

1. A method for determining a cardiomyocyte manganese ion uptake rate constant in one or more regions of interest in a heart of a subject comprising:
a providing step comprising:
providing a left ventricular myocardial image slice covering one or more regions of interest taken during a first time period;
providing one or more longitudinal relaxation time weighted images of the left ventricular blood pool, in the form of a saturation recovery imaging pulse sequence with a 90 degree flip angle and a short echo time, taken during the first time period to measure an average signal intensity in blood during the first time period;
providing a hematocrit measurement;
providing an extracellular volume measurement for each of the one or more regions of interest;
an administration step comprising administering a contrast agent comprising manganese 2+ ions;
an image acquisition step comprising:
performing image acquisition of the left ventricular blood pool using a longitudinal relaxation time weighted pulse sequence, in the form of a saturation recovery pulse sequence with a 90 degree flip angle and a short echo time at a plurality of time intervals during a second time period to measure respective signal intensities in blood at the plurality of time intervals;
performing a longitudinal relaxation time map image acquisition of a left ventricular myocardial image slice covering the one or more regions of interest during a third time period;
a measurement step comprising measuring a signal intensity in blood at each of the plurality of time intervals using an image taken at a time point within said corresponding time interval; and
a calculation step comprising:
calculating a change in a myocardial longitudinal relaxation rate for each region of interest from a baseline myocardial longitudinal relaxation rate extracted from the myocardial image slice taken during the first time period and a myocardial longitudinal relaxation rate at an end time point of an MRI examination extracted from the myocardial image slice during the third time period;
calculating a saturation recovery calibration constant from the saturation recovery imaging pulse sequence taken during the first time period and the average signal intensity in the blood, wherein calculating the saturation recovery calibration constant at the region of interest comprises using the equation:
$${k}_{S R} = \frac{{SI}_{b l o o d} ( {t}_{B L} )}{( 1 - {e}^{- T D / T {1}_{b l o o d} ( {t}_{B L} )} )}$$
wherein SI_{blood}(t_{BL}) is the average signal intensity in blood at the first time period, TD is the trigger delay, and T1_{blood}(t_{BL}) is the longitudinal relaxation time in blood taken during the first time period; and
calculating a blood longitudinal relaxation rate for each of the plurality of time intervals using respective measured signal intensities in blood at the plurality of time intervals and a relationship between signal intensity and said plurality of relaxation rates according to the equation:
$$R {1}_{b l o o d} ( {t}_{i} ) = \frac{- ln ( 1 - \frac{{SI}_{b l o o d} ( {t}_{i} )}{{k}_{S R}} )}{T D}$$
wherein SI_{blood}(tᵢ) is the measured signal intensity in blood at time interval tᵢ, k_{SR} is the saturation recovery calibration constant, and TD is trigger delay;
calculating a change in blood longitudinal relaxation rate for each of the plurality of time intervals and for the MRI examination end timepoint;
integrating over all of the changes in blood longitudinal relaxation rate for each of the plurality of time intervals; and
using the integral over all the changes in blood longitudinal relaxation rate in conjunction with the myocardial longitudinal relaxation rate at the end time point in a proportionality relationship with a cardiomyocyte Mn²⁺ uptake rate constant to estimate the cardiomyocyte Mn²⁺ uptake rate constant for each of the one or more regions of interest;
wherein said first time period is before an administration of the contrast agent, said second time period is during and after an administration of the contrast agent, and said third time period encompasses the end timepoint of an MRI examination.

2. The method of claim 1, wherein performing image acquisition of the left ventricular blood pool consists of taking a single image per time interval.

3. (canceled)

4. The method of claim 1, further comprising using the estimated cardiomyocyte Mn²⁺ uptake rate constant for each region of interest to indicate a T-tubule density at said corresponding region of interest.

5. The method of claim 1, wherein the one or more regions of interest comprise a plurality of regions of interest across a left ventricular myocardium of the heart, and wherein the estimated cardiomyocyte Mn²⁺ uptake rate constant for each of the plurality of regions of interest provides a t-tubule density estimate map across said left ventricular myocardium.

6. The method of claim 1, wherein the hematocrit, the ECV in the at least one region of interest and the integral over all the changes in blood relaxation for each of the plurality of time intervals are used in the following equation:
$${K}_{Cell}^{Mn} = \frac{Δ R {1}_{ROI} - ECV * \frac{Δ R {1}_{blood}}{( 1 - hct )}}{( 1 - ECV ) * \frac{{\int }_{0}^{{t}_{E}} Δ R {1}_{blood} ( t ) dt}{( 1 - hct )}}$$
wherein ΔR1_{ROI} is the change in myocardial longitudinal relaxation rate in the region of interest, ΔR1_{blood} is the change in blood longitudinal relaxation rate, ECV is the extracellular volume fraction for the region of interest, hct is the hematocrit, and
$${\int }_{0}^{{t}_{E}} Δ R {1}_{b l o o d} ( t ) d t$$
is the integral over the change in blood longitudinal relaxation rate from the second time period to the third time period;
to estimate of the cardiomyocyte Mn²⁺ uptake rate constant.

7. The method of claim 1, wherein providing at least one left ventricular image slice in the form of a longitudinal relaxation time map taken during a first time period, and/or performing acquisition of myocardial image slices using a longitudinal relaxation time map comprises using a modified look-locker inversion recovery longitudinal relaxation time map.

8. The method of claim 1, wherein each image in the saturation recovery imaging pulse sequence to measure the average signal intensity in the blood is obtained using:
a 90 degree flip angle;
an echo time less than 1 millisecond; and
a trigger delay in the range of 150 to 1000 milliseconds.

9. The method of claim 1, wherein the contrast agent comprising manganese 2+ ions has a log stability constant of 10 to 18.

10. (canceled)

11. A system for determining a cardiomyocyte manganese 2+ ion uptake rate constant in one or more regions of interest in a heart of a subject comprising:
a database module;
a measuring module;
a calculation module; and
computer readable media comprising instructions that, when executed on a processor, perform the tasks of:
determining a cardiomyocyte manganese 2+ ion uptake rate constant in one or more regions of interest in a heart of a subject by:
a) retrieving a myocardial image slice covering the one or more regions of interest at a first time period to retrieve a baseline myocardial longitudinal relaxation rate;
b) retrieving a myocardial image slice covering the one or more regions of interest at a third time period to retrieve an examination end time myocardial longitudinal relaxation rate; and
c) calculating a change in a myocardial longitudinal relaxation rate at each region of interest using the baseline myocardial longitudinal relaxation rate and the examination end time myocardial longitudinal relaxation rate at respective regions of interest; and
d) retrieving a longitudinal relaxation time map of left ventricle blood pool from the first time period to measure a baseline longitudinal relaxation time in blood;
e) retrieving an average signal intensity in the blood from the first time period, from a saturation recovery pulse sequence; and
f) accessing a pre-performed left ventricular imaging slice covering the left ventricular blood pool using a Saturation Recovery pulse sequence with a 90 degree flip angle and a short echo time at the plurality of time intervals;
g) measuring a signal intensity in blood at each of the plurality of time intervals from Saturation Recovery pulse sequence image taken at a respective time interval;
h) calculating a saturation recovery calibration constant at the region of interest using the equation:
$${k}_{S R} = \frac{{SI}_{b l o o d} ( {t}_{B L} )}{( 1 - {e}^{- T D / T {1}_{b l o o d} ( {t}_{B L} )} )}$$
wherein SI_{blood}(t_{BL}) is the average signal intensity in blood at the first time period, TD is the trigger delay, and T1_{blood}(t_{BL}) is the longitudinal relaxation time in blood taken during the first time period.
i) calculating a blood longitudinal relaxation rate for each of the plurality of time intervals using the retrieved signal intensity in blood at the corresponding plurality of time intervals, and a relationship between signal intensity and said plurality of relaxation rates according to the equation:
$$R {1}_{b l o o d} ( t ) = \frac{- ln ( 1 - \frac{{SI}_{b l o o d} ( t )}{{k}_{S R}} )}{T D}$$
wherein SI_{blood}(t) is a signal intensity in blood at the plurality of time intervals, k_{SR} is the saturation recovery calibration constant, and TD is trigger delay; and
j) retrieving a blood longitudinal relaxation rate at the first time period; and
k) calculating a change in blood longitudinal relaxation rate for each of the plurality of time intervals using the blood longitudinal relaxation rate for each of the plurality of time intervals and the blood longitudinal relaxation rate at the first time period;
l) integrating over the change in blood longitudinal relaxation rate for all time intervals; and
m) retrieving a hematocrit value;
n) retrieving an extracellular volume for the region of interest; and
o) calculating the manganese 2+ ion cellular uptake rate constant using a proportionality relationship between:
the change in myocardial longitudinal relaxation rate at the region of interest;
the change in blood longitudinal relaxation rate;
the hematocrit;
the extracellular volume at the region of interest; and
the integral of the change in blood longitudinal relaxation rate for all time intervals;
wherein the first time period is before an administration of a contrast agent comprising manganese 2+ ions, the second time period is during and after the administration of the contrast agent, and the third time period encompasses an end time in the examination;
wherein the said computer readable media is distributed across at least the measuring module and the calculation module of the system;
wherein the computer readable media distributed in the measuring module is configured, when executed on the processor, to perform at least task g); and
wherein the computer readable media distributed in the calculation module is configured, when executed on the processor, to perform at least task h), i), k) and o).

12. (canceled)

13. The system of claim 23, wherein retrieving a myocardial image slice at the region of interest at the third time period comprises:
accessing a pre-performed myocardial image slice taken during the third time period; or
performing, by the MRI scanner, a longitudinal relaxation map covering all of the one or more regions of interest during the third time period.

14. The method of claim 1, wherein calculating a change in a myocardial longitudinal relaxation rate at the region of interest further comprises using the equation:
$$Δ R {1}_{ROI} ( {t}_{E} ) = \frac{1}{T {1}_{ROI} ( {t}_{E} )} - \frac{1}{T {1}_{ROI} ( {t}_{B L} )}$$
wherein ΔR1_{ROI}(t_{E}) is the change in myocardial longitudinal relaxation rate at the region of interest at the end time of the MRI examination, T1_{ROI}(t_{E}) is the longitudinal relaxation time at the region of interest taken at the examination end time, and T1_{ROI}(t_{BL}) is the longitudinal relaxation time at the region of interest taken during the first time period.

15. (canceled)

16. (canceled)

17. The method of claim 1, wherein wherein calculating a change in blood longitudinal relaxation rate for each of the plurality of time intervals comprises using the equation:
$$Δ R {1}_{b l o o d} ( {t}_{i} ) = R {1}_{b l o o d} ( {t}_{i} ) - R {1}_{b l o o d} ( {t}_{B L} )$$
wherein R1_{blood}(tᵢ) is the blood longitudinal relaxation rate for each of the plurality of time intervals tᵢ and R1_{blood}(t_{BL}) is blood longitudinal relaxation rate at the first time period.

18. The method of claim 1, wherein calculating the manganese ion cellular uptake rate constant comprises using the equation:
$${K}_{Cell}^{Mn} = \frac{Δ R {1}_{ROI} - ECV * \frac{Δ R {1}_{blood}}{( 1 - hct )}}{( 1 - ECV ) * \frac{{\int }_{0}^{{t}_{E}} Δ R {1}_{blood} ( t ) dt}{( 1 - hct )}}$$
wherein ΔR1_{ROI} is the change in myocardial longitudinal relaxation rate in the region of interest, ΔR1_{blood} is the change in blood longitudinal relaxation rate, ECV is the extracellular volume for the region of interest and hct is the hematocrit.

19. Use of a contrast agent containing manganese 2+ ions in the method of claim 1 to determine a cardiomyocyte manganese ion uptake rate constant in one or more regions of interest in a heart of a subject.

20. The system of claim 11, wherein calculating a change in a myocardial longitudinal relaxation rate at the region of interest further comprises using the equation:
$$Δ R {1}_{ROI} ( {t}_{E} ) = \frac{1}{T {1}_{ROI} ( {t}_{E} )} - \frac{1}{T {1}_{ROI} ( {t}_{B L} )}$$
wherein ΔR1_{ROI}(t_{E}) is the change in myocardial longitudinal relaxation rate at the region of interest at the end time of the MRI examination, T1_{ROI}(t_{E}) is the longitudinal relaxation time at the region of interest taken at the examination end time, and T1_{ROI}(t_{BL}) is the longitudinal relaxation time at the region of interest taken during the first time period.

21. The system of claim 11, wherein calculating a change in blood longitudinal relaxation rate for each of the plurality of time intervals comprises using the equation:
$$Δ R {1}_{b l o o d} ( {t}_{i} ) = R {1}_{b l o o d} ( {t}_{i} ) - R {1}_{b l o o d} ( {t}_{B L} )$$
wherein R1_{blood}(tᵢ) is the blood longitudinal relaxation rate for each of the plurality of time intervals tᵢ and R1_{blood}(t_{BL}) is blood longitudinal relaxation rate at the first time period.

22. The system of claim 11, wherein calculating the manganese ion cellular uptake rate constant comprises using the equation:
$${K}_{Cell}^{Mn} = \frac{Δ R {1}_{ROI} - ECV * \frac{Δ R {1}_{blood}}{( 1 - hct )}}{( 1 - ECV ) * \frac{{\int }_{0}^{{t}_{E}} Δ R {1}_{blood} ( t ) dt}{( 1 - hct )}}$$
wherein ΔR1_{ROI} is the change in myocardial longitudinal relaxation rate in the region of interest, ΔR1_{blood} is the change in blood longitudinal relaxation rate, ECV is the extracellular volume for the region of interest and hct is the hematocrit.

23. The system of claim 11, further comprising an MRI scanner (MRI), wherein the pre-performed left ventricular imaging slice covering the left ventricular blood pool is acquired by the MRI scanner (MRI) configured to acquire a saturation recovery pulse sequence of the left ventricular blood pool with a 90 degree flip angle and a short echo time at a plurality of time intervals during the second time period to measure respective signal intensities in blood at the plurality of time intervals.
